(19) United States
(12) Patent Application Publication
(10) Pub. No.: US 2004/0121324 A1
(43) Pub. Date: Jun. 24, 2004

(54) Barcoded synthetic lethal screening to identify drug targets

(76) Inventors: Charles M. Brenner, Philadelphia, PA (US); Daniel D. Shoemaker, San Diego, CA (US)

Correspondence Address: Pennie & Edmonds, 1155 Avenue of the Americas, New York, NY, 10036-2711, US

(21) Appl. No.: 10/182,209

(22) PCT Filed: Jan. 18, 2001
(86) PCT No.: PCT/US01/01661

(51) Int. Cl. C12Q 1/68; G06F 19/00; G01N 33/48; G01N 33/50; C12N 1/18; C12N 15/85; C12N 15/74

(52) U.S. Cl. 435/6; 435/325; 435/254.2; 435/455; 435/483; 702/20

(57) ABSTRACT

The present invention relates to methods of using synthetic lethal screening techniques to identify drug targets. The methods of the present invention use “barcoded” libraries of cells, where the library consists of a collection of different mutant clones, each mutant clone bearing a knock-out mutation of a different gene. Each mutant clone has a unique DNA identifier tag, or “barcode,” to allow for quick and convenient identification of the clone and its mutation. The use of such a library allows for rapid, quantitative, sensitive and simple identification of genes which interact with a mutated target gene. So identified genes are promising targets for drug screening.

### 1. INTRODUCTION

The present invention relates to methods of using synthetic lethal screening techniques to identify drug targets. The methods of the present invention use “barcoded” libraries of cells, where the library consists of a collection of different mutant strains, each mutant strain bearing a knock-out mutation of a different gene. Each mutant strain also has a unique DNA identifier tag, or “barcode,” to allow for quick and convenient identification of the clone and its mutation. The use of such a library allows for rapid and simple identification of genes which interact with a mutated target gene. So identified genes are promising targets for drug screening.

### 2. BACKGROUND OF THE INVENTION

Among the mechanisms thought to be involved in the development of cancer is the activation of oncogenes and the loss of tumor suppressor genes (TSGs). The molecular medicine of the 21st century will depend on genetic diagnosis of patients' tumors, followed by application of specific chemotherapeutics tailored to tumors with similar genetic changes. It is expected that such compounds will be less generally cytotoxic, more antineoplastic, and more effective in curing cancer while maintaining a high quality of life. When oncogenes are activated in tumors, their protein products and the proteins that modify them present obvious molecular targets for the design and optimization of new-generation antineoplastics. For example, tumors with activated or amplified HER2 genes are hypersensitive to Her2-directed drugs. However, many cancers are characterized by losses of genetic information at loci including p53, p16, PTEN, APC and FHIT. Unfortunately, losses of specific TSGs do not immediately suggest cellular targets, inhibition of which would kill cells with these inactivated genes. It would be desirable to have a method capable of identifying such cellular targets.

2.1. Synthetic Lethal Screening

The traditional yeast synthetic lethal screens use a “plasmid shuffling” strategy (Sikorski R S, Boeke, 1991, Methods Enzymol., 194:302-18, incorporated by reference in its entirety). The first step involves constructing a strain where the target gene has been deleted. This gene is then re-introduced into the cell oil a low-copy plasmid that also contains a marker (e.g., ADE2) that can be used to select for the plasmid or detect the loss of the plasmid. The strain is then mutagenized and allowed to grow in the absence of selection for the plasmid. Under this condition, the majority of strains will lose the plasmid over time which results in colonies with a red and white sectored appearance (yeast strains missing the ADE2 generate red colonies due to the accumulation of an intermediate). However, a cell that has acquired a mutation in a gene that is synthetically lethal with the target gene will generate a colony that is completely white. The colonies are white because any cells that lose the plasmid die due to the lethal nature of the double mutant.

Another approach for performing synthetic lethal screens in yeast involves generating a conditional lethal mutant for a target gene. The strain bearing such a mutation is then mutagenized and screened for second-site mutations that specifically exacerbate its temperature sensitivity. This approach has successfully been used to identify proteins involved in the translocation step of protein secretion (Boisrame A, et al., 1999, Mol Gen Genet, 261(4-5):601-9, incorporated by reference in its entirety).

The disadvantages of the traditional approach are:

1. Only strong synthetic phenotypes can be detected due to the lack of sensitivity of the colony-sectoring assay.

2. The mutant hunts in synthetic lethal screens are limited by the mutagenic agent that is being used to generate the mutations. For example, not all genes can be disrupted using methyl methane sulfonate, which non-specifically methylates DNA.

3. Requires a genetic system such as yeast that has marker genes that can be used to select for or detect the loss of a plasmid.

4. Identification of the synthetically lethal mutated gene in the genome is very labor intensive.

### 3. SUMMARY OF THE INVENTION

The present invention relates to methods of using synthetic lethal screening techniques to identify drug targets. The methods of the present invention entail the use of “barcoded” libraries of cells, where the library consists of a collection of different mutant clones, each mutant clone bearing a knock-out mutation of a different gene. Additionally, each mutant clone has a unique DNA identifier tag, or “barcode,” to allow for quick and convenient identification of the clone and its mutation. The library of mutant clones is used as a panel of “secondary mutations,” against which the effects of a “primary mutation” can be assessed. The “primary mutation” is a knock-out mutation induced in a particular target gene in each of the clones comprising the barcoded library. After inducing the primary mutation in the mutant clones of the library, the interaction of the primary mutation with each of the secondary mutations present in the library can be determined. After inducing the primary mutation in the library, the library is allowed to grow. After several doublings of the library, those clones harboring secondary mutations which interact with the primary mutation causing a decrease in the growth rate of said clones will be selected against, i.e., present at a lower concentration relative to clones not harboring such interacting secondary mutations. Because each mutated clone is tagged (barcoded), the relative abundance of each clone can be easily determined by assaying for each of the tags. This may be done, for example, by hybridizing DNA obtained from the culture to a DNA microarray consisting of DNA molecules complementary to each tag. Missing tags represent those clones that harbor a “synthetic lethal” secondary mutation, i.e., a mutation that interacts with a primary mutation resulting in decreased rate of growth of the cell harboring both the primary and the secondary mutation. Because the library of mutants is tagged and characterized, identification of under-represented tags in the library after introduction of the primary mutation is tantamount to identifying a gene product which, if knocked out, causes a decreased growth rate when combined with the primary mutation. Such gene products are excellent candidates for use in drug screening protocols designed to identify agents capable of inhibiting the growth of cells harboring the primary mutation. This is so because the screen of the invention identifies genes that, if knocked out, cause decreased growth rates of cells also harboring the primary mutation.

Citation or discussion of a reference hereinabove shall not be construed as an admission that such is prior art to the present invention.

### 4. DETAILED DESCRIPTION OF THE INVENTION

The present invention relates to the identification of genes differentially required for the survival of mammalian cells missing a target gene. The target gene may be, as a non-limiting example, any of a class of genes including tumor suppressor genes and mutator genes, the function of which is absent or reduced in cancer cells. This invention relates to use of synthetic lethal screening to identify genes that are more important to the growth or survival of cells missing a particular target gene as compared to how important those genes are for the growth or survival of wild-type cells with the target gene. This information is used to rationalize a drug target in mammalian cells of defined genotype. Given a cell with an inactivating mutation in its version or homolog of the target gene, a synthetic lethal screen might identify genes X, Y and Z in that cell, each of which have a more deleterious phenotype as double mutants with the target gene mutants than as single mutants. Upon identification of gene products X, Y and Z (or their homologs) in mammals, these gene products would be rationalized as drug targets for the elimination of cells missing the target gene. Additionally, this invention relates to the use of barcoded libraries and oligonucleotide arrays to perform synthetic lethal screens. The use of libraries consisting of barcoded mutants has been extensively described (Giaever, G., et al., 1999, Nat Genet 21(3):278-83; Shoemaker, D. D., et al., 1996, Nat Genet 14(4):450-6; Winzeler, E. A., et al., 1999, Science 1999, 285(5429):901-6; Hensel, M., et al., 1995, Science 269(5222):400-3).

Performing synthetic lethal screens with a library of bar-coded deletion strains offers several advantages over the traditional methods that have been developed in yeast.

1. The quantitative nature of the competitive growth studies with bar-coded deletion mutants allows subtle differences in growth rates to be detected. This makes it possible to detect synthetic lethal combinations that would normally be undetectable in the standard synthetic lethal mutations. For example, a strain with 5% growth difference will be depleted from the population by 50% after 20 population doublings.

2. It is possible to generate a perfect library of bar-coded deletion mutants. Every gene in the genome can be deleted and each deletion removes the entire coding region of the target gene.

3. Once a collection of bar-coded cell lines has been generated, it is relatively easy to perform synthetic lethal screens with different primary mutations. The collection of strains bearing potential secondary mutations only has to be generated a single time. This is in contrast to the traditional approach where potential secondary mutations must be regenerated every time a new primary mutation is analyzed. This fact is especially important in diploid organisms where both copies of each gene must be disrupted to see a phenotype.

Definition of Terms

Synthetically Lethal

When two non-lethal mutations (a non-lethal mutation is a mutation with little or no effect on cell viability or growth) are present in the same cell and together cause the cell to be unable to grow, or to grow at a lower rate or are lethal to the cell, the combined mutations are considered to be synthetically lethal mutations, i.e., the combination of the two mutations is detrimental to the viability of a cell bearing that combination.

Primary Mutation and Secondary Mutation

In a pair of synthetically lethal mutations, one of the mutations is termed the primary mutation, and the other is termed the secondary mutation. The primary mutation is the mutation of the target gene, i.e., the gene for which one wishes to identify secondary mutations which are synthetically lethal. The mutations present in the strains comprising the mutation library are the secondary mutations. The methods of the present invention provide a way of determining which of the secondary mutations present in a library are synthetic lethal mutations with respect to a given primary mutation.

Knock-Out Mutation

A knock out mutation of a gene causes the loss of function of the mutated gene. This is generally the result of a disruption or deletion of the gene.

Barcoded Mutation Library

A barcoded mutation library is a library comprised of different mutant strains of cells, each strain bearing a unique DNA tag. This DNA tag is referred to as the barcode. The DNA tags are identifiable by, for example, utilizing the polymerase chain reaction to amplify the tags and then hybridizing the amplified tags to a DNA micro-array comprised of sequences complementary to each tag. An example of such a library is a collection of yeast deletion mutants, each mutant having a different open reading frame (ORF) deleted and having in its genome one or more unique DNA tags.

The present invention provides methods of detecting synthetic lethality caused by the interaction of a primary and a secondary mutation. A preferred method comprises the steps of:

(a) introducing a primary mutation into one or more cells present in a library of cells having a secondary mutation in a second gene, said library comprising a population of cells wherein each cell in said population has a different secondary mutation in a different gene;

(b) incubating the cells of step (a) under conditions which would, in the absence of step (a), allow the cells to grow; and

(c) comparing the growth of each cell of step (b) that has the primary mutation with the growth of a control cell without the primary mutation but containing said secondary mutation,

(d) identifying any cell in step (b) that exhibits a decreased rate of growth as compared to the rate of growth of a control cell without the primary mutation but containing said secondary mutation as containing a secondary mutation that causes a decreased rate of growth when combined with the primary mutation in a cell; and

(e) determining in which gene the secondary mutation that causes a decreased rate of growth when combined with the primary mutation identified in step (d) resides.

In a preferred embodiment, the method of the invention comprises the additional step of

(f) isolating the gene in which the secondary mutation that causes a decreased rate of growth when combined with the primary mutation identified in step (d) resides.

When the mutation library is not a human cell mutation library, the method further comprises the step of:

(g) isolating the human homolog of the gene isolated in step (f).

The invention also provides a barcoded deletion mutant library comprised of different mutant cells, each mutant cell having a different deletion mutation, wherein between 25% and 100%, 50% and 95%, or 60% and 90% of the cells comprising the library also have a primary mutation, wherein said primary mutation is a mutation of the same gene in each of the different mutant cells containing said primary mutation.

4.1. Barcoded Synthetic Lethal Screening in Yeast

In a preferred embodiment, yeast cells are used according to the methods of the invention. The following sections describe aspects of the claimed methods as they relate to the use of a yeast model system.

4.1.1 Yeast Mutation Libraries

Any library of mutants may be used as the panel of potential secondary mutations in which the primary mutation is induced according to the methods of the invention.

There are a number of methods well known in the art by a gene may be disrupted or mutated in yeast. In one embodiment, an entire gene and create a null allele, in which no portion of the gene is expressed. In other embodiments, a deletion allele may be constructed comprising only a portion of the gene which is not sufficient for gene function, which can be constructed, for example, by inserting a nonsense codon into the sequence of the gene such that translation of the mutant mRNA transcript ends prematurely. Alleles may also be made containing point mutations, individually or in combination, that reduce or abolish gene function. Such methods are well known in the art.

There are a number of different strategies for creating conditional alleles of genes. Broadly, an allele can be conditional for function or expression. An example of an allele that is conditional for function is a temperature sensitive mutation wherein the gene product is functional at one temperature (i.e., permissive temperature) but non-functional at a different temperature (i.e., non-permissive temperature), e.g., due to misfolding or mislocalization. One of ordinary skill in the art can produce mutant alleles which may have only one or a few altered nucleotides but which encode inactive or temperature-sensitive proteins. Temperature-sensitive mutant yeast cells express a functional protein at permissive temperatures but do not express a functional protein at non-permissive temperatures.

An example of an allele that is conditional for expression is a chimeric gene where a regulated promoter controls the expression of the gene. Under one condition the gene is expressed and under another it is not. One may replace or alter the endogenous promoter of the gene with a heterologous or altered promoter that can be activated only under certain conditions. These conditional mutants only express the gene under defined experimental conditions. All of these methods are well known in the art. For example, see Stark, 1998, Methods in Microbiology 26:83-100; Garfinkel et al., 1998, Methods in Microbiology 26:101-118; and Lawrence & Rothstein, 1991, Methods in Enzymology 194:281-301.

In another embodiment of the invention, a gene may have decreased expression without disrupting or mutating the gene. For instance, the expression of gene may be decreased by transforming yeast with an antisense molecule under the control of a regulated or constitutive promoter (see Nasr et al., 1995, Molecular & General Genetics 249:51-57). Such an antisense construct operably linked to an inducible promoter and introduced into S. cerevisiae to study the function of a conditional allele (see Nasr et al. supra), or to act as a perturbations of a cell.

Gene expression may also be decreased by inserting a sequence by homologous recombination into or next to the target gene wherein the sequence targets the mRNA or the protein for degradation. For instance, one can introduce a construct that encodes ubiquitin such that a ubiquitin fusion protein is produced. This protein will be likely to have a shorter half-life than the wild type protein. See, e.g., Johnson et al., 1992, EMBO J. 11:497-505.

In a preferred mode, a target gene is completely disrupted in order to ensure that there is no residual function of the gene. One can disrupt a gene by “classical” or PCR-based methods. The “classical” method of gene knockout is described by Rothstein, 1991. However, in some embodiments, it is preferable to use a PCR-based deletion method because it is faster and less labor intensive.

Preferably, the mutation library is a deletion mutation library and is barcoded. Barcoded deletion strains have been generated by the Saccharomyces Genome Deletion Project and described in several publications (Winzeler E A, et al., 1999, Science 285:901-6, Shoemaker, D., et al., 1996, Nature Genetics, 14, 450-456; Hensel, M., et al., 1995, Science 269(5222):400-3, each incorporated by reference in its entirety). The goal of Saccharomyces Genome Deletion Project is to generate a complete set of yeast deletion strains. A PCR-based gene deletion strategy (Baudin et al., 1993, Nucl. Acids Res. 21, 3329-3330; Wach et al., 1994, Yeast 10, 1793-1808, each incorporated by reference in its entirety) was used to generate individual deletion strains for each of the ˜6,200 open reading frames (ORFs) in the yeast genome. As part of the strain construction process, each deletion was uniquely tagged with two independent 20mer sequences. The presence of the tags can be detected via hybridization to a high-density oligonucleotide array, enabling growth phenotypes of the resulting deletion strains to be analyzed in parallel. More than 10,000 strains are currently available (Research Genetics, Huntsville, Ala.; American Type Culture Collection, Manassas, Va.).

To generate a pool of mutants, individual mutant strains can be grown to saturation in individual cultures of rich medium. The resulting cultures can be pooled together and used for the screens of the invention. In particular embodiments, the pool of mutants contains between 100 and 15,000 different mutant strains, between 1000 and 15,000 different mutant strains, between 1000 and 10,000 different mutant strains, or between 2000 and 8000 different mutant strains. The use of any number of strains is expressly contemplated in the present invention. In preferred embodiments, the library contains individual deletion strains for 100%, 90%, 80%, 70%, 60%, 50%, 40%, 30% or 20% of the open reading frames in the yeast genome.

4.1.2 Selection of Primary Mutations

The methods of the invention can be used to identify secondary mutations for any given primary mutation of a target gene. Preferred target genes include yeast homologs of any mammalian genes whose function is absent or reduced in cancer cells, e.g., tumor suppressor genes or mutator genes. Examples of such mammalian genes include p53, p16, PTEN, NF-1, NF-2, DPC4, MTS1, retinoblastoma gene, APC and FHIT.

It has been estimated that almost 2000 human genetic diseases have been recognized. The sequence of only 250 disease associated genes has been determined, and of these 250 genes, 105 bear similarity to yeast genes (Foury, F., 1997, Gene 195:1-10). Thus, there are many yeast target genes, the study of which may provide information useful for the treatment of human diseases.

For example, HNT2, the yeast homolog of the human tumor suppressor gene FHIT, may be used as the target gene (K. Huebner, et al., 1999, Advances in Oncology 15:3-10; U.S. Pat. No. 5,928,884, International Publication No. WO97/29119, incorporated by reference in its entirety). In the case of HNT2, the primary mutation would be a mutation that causes the reduction or loss of the HNT2 gene product or gene product function. An example of such a primary mutation would be an HNT2 knock-out mutation, where the sequence of HNT2 is deleted and, optionally, replaced with another gene, for example, a marker gene such as URA3. If using an HNT2 knock-out primary mutation results in the identification of secondary mutations in a yeast mutation library, human homologs of the yeast genes associated with those secondary mutations are potential drug screening targets useful for the identification of compounds able to inhibit the growth of tumor cells containing reduced or non-existent levels of the tumor suppressor FHIT.

While homologs of mammalian tumor suppressor genes are preferred genes of interest, the primary mutation can be a mutation of any gene. For example, each individual mutation present in a yeast barcoded deletion mutation library may be used as a primary mutation to identify which, if any of the other mutations present in the library can provide synthetic lethality in combination with that primary mutation. Thus, the screening of all possible combinations of two mutants present in a given collection of barcoded deletion mutants for synthetic lethality is also provided by the present invention.

4.1.3 Introduction of a Primary Mutation into a Yeast Mutation Library

The primary mutation of the target gene may be introduced into the yeast mutation library by any means known to one of skill in the art, including those methods provided in section 5.1.2. Preferably, the primary mutation is introduced into a high percentage of the cells present in the library, preferably greater than 30%, greater than 40%, greater than 50%, greater than 60%, greater than 70%, greater than 80%, greater than 90% or greater than 95% of the cells of the mutation library receive the primary mutation. The goal is to generate enough independent isolates such that most or all of the possible double mutants are produced. The use of a yeast mutation library provides several advantages. The yeast genome readily undergoes homologous recombination, allowing for targeted mutation of the target gene. In addition, yeast cells can be propagated as either haploid or diploid cells, so only one copy of the target gene need be mutated.

In a preferred embodiment, the primary mutation is introduced into the mutation library by mating the cells of the mutation library with a cell bearing the primary mutation. This approach ensures that enough independent isolates can be tested to maintain the complexity of the mutation library. The cells of the mutation library can be placed into separate mating reactions with cells bearing the primary mutation, as well as with control cells identical to the cells bearing the primary mutation, but lacking the primary mutation. In a preferred embodiment, the cells bearing the primary mutation and the cells of the mutation library have different selectable markers, allowing for selection of diploid cells, i.e., cells with both selectable markers. The control cell should also contain the same selectable marker as does the cell bearing the primary mutation. For any given double mutant cell containing both a primary and a secondary mutation, the control cell corresponding to that double mutant is a cell containing only the secondary mutation. In a preferred embodiment, the two selectable markers are URA3 and KanMX. In a more preferred embodiment, the URA3 gene is used to create the primary mutation by homologous recombination with the target gene, causing replacement of the target gene with the URA3 gene. In this preferred embodiment, the mutations in the cells of the mutation library are produced by homologous recombination with the KanMx gene, rendering them resistant to G418. A variety of other selectable markers are available (Goldstein, A. L., et al., 1999, Yeast, 15(6):507-11). In this preferred embodiment, diploids are selected for by incubating the mated cells in the presence of G418 and SC-URA media.

The diploid cells obtained from the above mating can then be incubated and made to undergo sporulation, producing haploid cells. Preferably, only those double mutant haploid cells with both the primary mutation and a secondary mutation are obtained. In a preferred embodiment, the secondary mutations and the primary mutation are created by homologous recombination with different selectable markers, allowing for the selection of cells containing both markers and, consequently, both mutations. To ensure that only haploid cells are present, it is preferable that either the cells of the mutation library or the cells bearing the primary mutation (but not both) have a dominant selectable marker. An example of such a dominant selectable marker is the Can1 gene (Broach, J. R., et al., 1979, Gene 8(1):121-33). Any cell with a functional copy of the Can1 gene is sensitive to canavanine. Another example is the CYH2 gene; any cell with a copy of the CYH2 gene is sensitive to cycloheximide. Any diploid cell, which would have to contain a copy of the Can1 gene since the diploid cell was produced by the mating of one cell with the Can1 gene and one without the Can1 gene, would be selected against in the presence of canavanine, leaving only haploid cells.

Another preferred method of introducing the primary mutation into the mutation library is by direct transformation. This approach offers the advantage of requiring viewer steps compared to the above-described mating strategy. Specifically, the genomic regions flanking the target gene are cloned onto each side of a selectable marker gene, such as the URA3 gene. This deletion construct is introduced into the mutation library by homologous recombination. After selection for the marker, only cells which have successfully integrated the selectable marker into the genome can survive. In yeast, over 90% of the insertions will be into the targeted gene (Wach, A., 1996, Yeast, 12(3):259-65). For a control, a deletion construct can be generated that contains the selectable marker flanked by targeting homology to a gene not necessary for growth of the cell, such as the HO endonuclease gene (YDL227c). This gene is a good control because the gene product is not required for normal vegetative growth (Baganz, F., et al., 1997, Yeast 13:1563-1573, incorporated by reference in its entirety).

4.1.4 Detection of Synthetic Lethality

The presence of synthetic lethality between the primary mutation and any of the mutation present in the mutation library can be detected by any means know to those of skill in the art. Preferably, after introduction of the primary mutation into the mutation library and selection for those cells containing both the primary mutation and a secondary mutation, the resulting double mutant haploid cells are allowed to reproduce. Preferably, the cells are grown under conditions and for sufficient time to allow for at least 5, at least 10, at least 15, 20, about 20, at least 20, at least 30, at least 40, at least 50, 60, about 60, or at least 60 population doublings. This competitive outgrowth period allows for the amplification of any differential growth rates of the double mutants. For example, if a cell in a population of cells grows at a rate only 5% slower than the other cells of the population, that cell will be depleted from the population by 50% as compared to the other cells of the population after 20 population doublings. Preferably, a control experiment is also be performed in which a collection of single mutant controls (i.e., cells bearing only the secondary mutation) is grown under the same conditions for the same amount of time.

Following the outgrowth, the abundance of double mutant cells bearing each secondary mutation is determined. The abundance of each double mutant is compared to the abundance of the single mutant control cell bearing the same secondary mutation. If a double mutant and its corresponding single mutant control grow at the same rate (i.e., there is little or no difference between the abundance of the double mutant and the abundance of the single mutant control), then no synthetic lethality between those two genes was detected, and the secondary mutation is not a synthetic lethal mutation with respect to the primary mutation. However, if a double mutant grows at a slower rate compared to its corresponding single mutant control (i.e., there fewer, or undetectable amounts of the double mutant as compared to the corresponding single mutant), then synthetic lethality between those two genes was detected, and the secondary mutation is a synthetic lethal mutation with respect to the primary mutation.

In a preferred embodiment, the relative amounts of the double and corresponding single mutants are determined by detecting a DNA tag, or barcode, present in the mutation library. A barcoded mutation library, described above, allows for rapid and simple assessment of the population of mutation bearing cells. Because each member of a barcoded mutation library has a unique DNA tag, and because each tag is associated with a known deletion mutation, detection (or lack thereof) of a given DNA tag allows one to know which gene is deleted in the cell that bore that tag. In addition, the tags can be detected by first amplifying and fluorescently labeling them via the polymerase chain reaction, followed by hybridization of the amplified products with a DNA microarray comprised of DNA molecules complementary to the DNA tags. Examples of suitable fluorescent labels include Cy3, Cy5 and fluorescein. The use of a DNA microarray to detect tags present in a barcoded yeast mutation library has been published (Winzeler, E. A., et al., 1999, 285:901-906, incorporated by reference in its entirety). Such arrays can be produced by one of skill in the art according to established protocols (Marton, M. J., et al., 1998, Nat Med 4(11):1293-301) or obtained commercially (Affymetrix Inc., Santa Clara, Calif., see also U.S. patent application Ser. No. 09/303,082, filed Apr. 30, 1999). Each address of the DNA microarray contains DNA complementary to a known DNA tag. After hybridization, the amount of fluorescence detectable at a given location in the DNA microarray reveals the relative abundance of the cell bearing the tag complementary to the DNA at that location. Preferably, the tags in the single mutant control cells are amplified and labeled with a fluorophore different from the one used to label the amplified tags from the double mutant cells. When the differently labeled tags are simultaneously hybridized to the DNA microarray, the ratio of one fluor to the other detectable at each address of the DNA microarray provides a direct measure of the relative abundance of each double mutant with respect to its corresponding single mutant that was present in the culture from which the tags were amplified. Ratios close to 1:1 indicate that there is no difference in the growth rates of the single and double mutant, while ratios varying significantly from 1:1 indicate that the single and double mutants grow at different rates. Preferably, a ratio of single mutant fluorophore to double mutant fluorophore of greater than 2:1, greater than 3:1, greater than 5:1, greater than 8:1, or greater than 10:1 is considered to be an indicator of synthetic lethality in the screening methods of the invention.

Optionally, one may wish to identify those double mutants which are present at a reduced level compared to the other double mutants instead of compared to a control cell. The cells present at a lower abundance as compared to the other double mutant cells is identified as a cell bearing a primary mutation and a synthetic lethal secondary mutation.

4.2. Barcoded Synthetic Lethal Screening in Mammalian Cells

4.2.1 Mammalian Cell Mutation Libraries

Any library of mutants may be used as the panel of secondary mutations in which the primary mutation is introduced according to the methods of the invention. Mutants strains comprising the mutation library may be constructed by any method known to those of skill in the art. Preferably, each mutant strain exhibits a reduction or absence of the expression of a different gene. The reduced level of gene expression or activity can be generated by deleting or mutating at least one copy of the gene, by expressing a dominant negative form of a component of a cellular pathway of the gene, or by lowering the activity or abundance of the RNA encoded by the gene. The activity or abundance of a gene encoded RNA can be lowered by means of a ribozyme, an anti-sense nucleic acid, a double-stranded RNA or an aptamer.

Underexpression of a protein in tissue culture is best achieved by reducing the abundance or activity of the mRNA encoding that protein. Methods of reducing RNA abundance and activity currently fall within three classes, ribozymes, antisense species, and RNA aptamers (Good et al., 1997, Gene Therapy 4:45-54). Controllable exposure of a cell to these entities permits controllable perturbation of RNA abundances.

Ribozymes are RNAs which are capable of catalyzing RNA cleavage reactions. (Cech, 1987, Science 236:1532-1539; PCT International Publication WO 90/11364, published Oct. 4, 1990; Sarver et al., 1990, Science 247:1222-1225). “Hairpin” and “hammerhead” RNA ribozymes can be designed to specifically cleave a particular target mRNA. Rules have been established for the design of short RNA molecules with ribozyme activity, which are capable of cleaving other RNA molecules in a highly sequence specific way and can be targeted to virtually all kinds of RNA. (Haseloff et al., 1988, Nature 334:585-591; Koizumi et al., 1988, FEBS Lett. 228:228-230; Koizumi et al., 1988, FEBS Lett. 239:285-288). Ribozyme methods for underexpression of a gene involve inducing expression in a cell, etc. of such small RNA ribozyme molecules. (Grassi and Marini, 1996, Annals of Medicine 28:499-510; Gibson, 1996, Cancer and Metastasis Reviews 15:287-299).

Ribozymes can be routinely expressed in vivo in sufficient number to be catalytically effective in cleaving mRNA, and thereby modifying mRNA abundances in a cell. (Cotten et al., 1989, EMBO J. 8:3861-3866). In particular, a ribozyme coding DNA sequence, designed according to the previous rules and synthesized, for example, by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter (e.g., a glucocorticoid or a tetracycline response element) is also introduced into this construct so that ribozyme expression can be selectively controlled. tDNA genes (i.e., genes encoding tRNAs) are useful in this application because of their small size, high rate of transcription, and ubiquitous expression in different kinds of tissues. Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly the abundance of virtually any RNA species in a cell can be perturbed.

In another embodiment, activity of an RNA (preferable mRNA) species, specifically its rate of translation, can be controllably inhibited by the controllable application of antisense nucleic acids. An “antisense” nucleic acid as used herein refers to a nucleic acid capable of hybridizing to a sequence-specific (e.g., non-poly A) portion of the RNA, for example its translation initiation region, by virtue of some sequence complementarity to a coding and/or non-coding region. The antisense nucleic acids of the invention are produced intracellularly by transcription of exogenous, introduced sequences in controllable quantities sufficient to perturb translation of the RNA.

Preferably, antisense nucleic acids are of at least six nucleotides and to about 200 oligonucleotides). The antisense nucleic acids of the invention comprise a sequence complementary to at least a portion of an RNA species. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a given RNA it may contain and still form a stable duplex (or triplex, as the case may be) with that RNA. One skilled in the art can ascertain a tolerable degree of mismatch by use of standard procedures to determine the melting point of the hybridized complex. The amount of antisense nucleic acid that will be effective in the inhibiting translation of a geven RNA can be determined by standard assay techniques.

The antisense nucleic acids of the invention are controllably expressed intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian and insect cells. Expression of the sequences encoding the antisense RNAs can be by any promoter known in the art to act in a cell of interest. Such promoters can be inducible or constitutive. Most preferably, promoters are controllable or inducible by the administration of an exogenous moiety in order to achieve controlled expression of the antisense oligonucleotide. Such controllable promoters include but are not limited to the Tet promoter, the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc.

Therefore, antisense nucleic acids can be designed to target virtually any mRNA sequence, and a cell can be routinely transformed with nucleic acids coding for such antisense sequences such that an effective and controllable amount of the antisense nucleic acid is expressed. Accordingly the translation of virtually any RNA species in a cell can be controllably perturbed.

Finally, in a further embodiment, RNA aptamers can be introduced into or expressed in a cell. RNA aptamers are specific RNA ligands for proteins, such as for Tat and Rev RNA (Good et al., 1997, Gene Therapy 4:45-54) that can specifically inhibit their translation.

To generate a pool of mutants, individual mutant strains can be grown to saturation in individual cultures of rich medium. The resulting cultures can be pooled together and used for the screens of the invention. In particular embodiments, the pool of mutants contains between 100 and 100,000 different mutant strains, between 1000 and 100,000 different mutant strains, between 10,000 and 50,000 different mutant strains, or between 20,000 and 50,000 different mutant strains. The use of any number of strains is expressly contemplated in the present invention. In preferred embodiments, the library contains individual deletion strains for 100%, 90%, 80%, 70%, 60%, 50%, 40%, 30% or 20% of the open reading frames in the mammalian cell genome.

Preferably, the mutation library is a deletion mutation library. Also, the mutation library is preferably barcoded. Barcoded deletion strains have been generated by the Saccharomyces Genome Deletion Project and described in several publications (Winzeler E A, et al., 1999, Science 285:901-6, Shoemaker, D., et al., 1996, Nature Genetics, 14, 450-456, each incorporated by reference in its entirety). The goal of Saccharomyces Genome Deletion Project is to generate a complete set of yeast deletion strains. A PCR-based gene deletion strategy (Baudin et al., 1993, Nucl. Acids Res. 21, 3329-3330; Wach et al., 1994, Yeast 10, 1793-1808, each incorporated by reference in its entirety) was used to generate individual deletion strains for each of the ˜6,200 open reading frames (ORFs) in the yeast genome. As part of the strain construction process, each deletion was uniquely tagged with two independent 20mer sequences. The presence of the tags can be detected via hybridization to a high-density oligonucleotide array, enabling growth phenotypes of the resulting deletion strains to be analyzed in parallel. More than 10,000 strains are currently available (Research Genetics, Huntsville, Ala.; American Type Culture Collection, Manassas, Va.).

4.2.2 Selection of Primary Mutations

The methods of the invention can be used to identify secondary mutations for any given primary mutation of a target gene. Preferred target genes include any mammalian genes whose function is absent or reduced in cancer cells, e.g., tumor suppressor genes or mutator genes. Examples of such mammalian genes include p53, p16, PTEN, NF-1, NF-2, DPC4, MTS1, retinoblastoma gene, APC and FHIT.

4.2.3 Introduction of a Primary Mutation into a Mammalian Cell Mutation Library

The primary mutation of the target gene may be introduced into the mammalian mutation library by any means known to one of skill in the art, including those provided in section 5.2.1 hereinabove. Preferably, the primary mutation is introduced into a high percentage of the cells present in the library, preferably greater than 30%, greater than 40%, greater than 50%, greater than 60%, greater than 70%, greater than 80% or greater than 90% of the cells of the mutation library receive the primary mutation. The result of the primary mutation is a reduced or nonexistent level of target gene expression or activity.

4.2.4 Detection of Synthetic Lethality

The presence of synthetic lethality between the primary mutation and any of the mutation present in the mutation library can be detected by any means know to those of skill in the art. Preferably, after introduction of the primary mutation into the mutation library and selection for those cells containing both the primary mutation and a secondary mutation, the resulting double mutant haploid cells are allowed to reproduce. Preferably, the cells are grown under conditions and for sufficient time to allow for at least 5, at least 10, at least 15, 20, about 20, at least 20, at least 30, at least 40, at least 50, 60, about 60, or at least 60 population doublings. This competitive outgrowth period allows for the amplification of any differential growth rates of the double mutants. For example, if a cell in a population of cells grows at a rate only 5% slower than the other cells of the population, that cell will be depleted from the population by 50% as compared to the other cells of the population after 20 population doublings. Preferably, a control experiment is also be performed in which a collection of single mutant controls (i.e., cells bearing only the secondary mutation) is grown under the same conditions for the same amount of time.

Following the outgrowth, the abundance of double mutant cells bearing each secondary mutation is determined. The abundance of each double mutant is compared to the abundance of the single mutant control cell bearing the same secondary mutation. If a double mutant and its corresponding single mutant control grow at the same rate (i.e., there is little or no difference between the abundance of the double mutant and the abundance of the single mutant control), then no synthetic lethality between those two genes was detected, and the secondary mutation is not a synthetic lethal mutation with respect to the primary mutation. However, if a double mutant grows at a slower rate compared to its corresponding single mutant control (i.e., there fewer, or undetectable amounts of the double mutant as compared to the corresponding single mutant), then synthetic lethality between those two genes was detected, and the secondary mutation is a synthetic lethal mutation with respect to the primary mutation.

In a preferred embodiment, the relative amounts of the double and corresponding single mutants are determined by detecting a DNA tag, or barcode, present in the mutation library. A barcoded mutation library, described above, allows for rapid and simple assessment of the population of mutation bearing cells. Because each member of a barcoded mutation library has a unique DNA tag, and because each tag is associated with a known deletion mutation, detection (or lack thereof) of a given DNA tag allows one to know which gene is deleted in the cell that bore that tag. In addition, the tags can be detected by first amplifying and fluorescently labeling them via the polymerase chain reaction, followed by hybridization of the amplified products with a DNA microarray comprised of DNA molecules complementary to the DNA tags. Examples of suitable fluorescent labels include Cy3, Cy5 and fluorescein. The use of a DNA microarray to detect tags present in a barcoded mutation library has been published (Winzeler, E. A., et al., 1999, 285:901-906, incorporated by reference in its entirety). Such arrays can be produced by one of skill in the art according to established protocols (Marton, M. J., et al., 1998, Nat Med 4(11):1293-301) or obtained commercially (Affymetrix Inc., Santa Clara, Calif.). Each address of the DNA microarray contains DNA complementary to a known DNA tag. After hybridization, the amount of fluorescence detectable at a given location in the DNA microarray reveals the relative abundance of the cell bearing the tag complementary to the DNA at that location. Preferably, the tags in the single mutant control cells are amplified and labeled with a fluorophore different from the one used to label the amplified tags from the double mutant cells. When the differently labeled tags are simultaneously hybridized to the DNA microarray, the ratio of one fluor to the other detectable at each address of the DNA microarray provides a direct measure of the relative abundance of each double mutant with respect to its corresponding single mutant that was present in the culture from which the tags were amplified. Ratios close to 1:1 indicate that there is no difference in the growth rates of the single and double mutant, while ratios varying significantly from 1:1 indicate that the single and double mutants grow at different rates. Preferably, a ratio of single mutant fluorophore to double mutant fluorophore of greater than 2:1, greater than 3:1, greater than 5:1, greater than 8:1, or greater than 10:1 is considered to be an indicator of synthetic lethality in the screening methods of the invention.

Optionally, one may with to identify those double mutants which are present at a reduced level compared to the other double mutants instead of compared to a control cell. The cells present at a lower abundance as compared to the other double mutant cells is identified as a cell bearing a primary mutation and a synthetic lethal secondary mutation.

4.3. Barcoded Synthetic Lethal Screening in Other Systems

It will be appreciated by one of skill in the art that the methods described above are readily adaptable to other systems such as bacterial cells, insect cells and plant cells. In preferred embodiments, the cells of the mutation library are C. elegans cells, drosophila cells, or E. coli cells.

### 5. EXAMPLE

### Barcoded Synthetic Lethal Screening Using a Yeast Deletion Mutation Library

Obtaining the Barcoded Mutation Library

The bar-coded deletion strains used in our synthetic lethal screens were generated by the Saccharomyces Genome Deletion Project (Winzeler E A, et al., 1999, Science 285:901-6, Shoemaker, D., et al., 1996, Nature Genetics, 14, 450-456). The goal of this project is to generate a complete set of yeast deletion strains. A PCR-based gene deletion strategy was used to generate individual deletion strains for each of the ˜6,200 ORFs in the yeast genome. As part of the strain construction process, each deletion was uniquely tagged with two independent 20mer sequences (Winzeler E A, et al., 1999, Science 285:901-6). The presence of the tags can be detected via hybridization to a high-density oligonucleotide array, enabling growth phenotypes of the resulting deletion strains to be analyzed in parallel. More than 10,000 strains are currently available through Research Genetics and the ATCC (Research Genetics, Huntsville, Ala.; American Type Culture Collection, Manassas, Va.). For the synthetic lethal experiment described below, a pool of 1,600 haploid strains (BY4739, MAT alpha leu2D0 lys2D0 ura3D0 CAN1 KanMX⁺) was used. These haploid alpha strains are resistant to the drug G418 and sensitive to the drug canavanine.

To generate the pool of bar-coded deletion mutants, each of the 1,600 strains were grown to saturation in individual 5-ml cultures of rich YPD (yeast extract-peptone-dextrose) medium. The resulting cultures were pooled together, glycerol was added to a final concentration of 15%, and 10 ml aliquots were made and stored at −80 degrees C.

Generating the Primary Mutation in the Target Gene

We are interested in the HNT2 gene because it is the yeast homolog of human FHIT, a human tumor suppressor gene which is deleted in many solid tumors (K. Huebner, et al., 1999, Advances in Oncology 15:3-10; U.S. Pat. No. 5,928,884, International Publication No. WO97/29119). Our goal is to identify new anti-cancer drug targets by identifying mutations that are synthetic lethal with HNT2. In this example, standard yeast genetic techniques were used to delete the HNT2 gene (YDR305c) in a haploid yeast strain (MATa can1 hnt2DO::URA3). Specifically, the entire coding region of the HNT2 gene was replaced with the selectable marker URA3 using homologous recombination. This strain contains a mutation in the CAN1 gene which confers resistance to the arginine analog canavanine. For a control, we generated an isogenic yeast strain that contains a functional copy of the HNT2 gene and the URA3 gene is inserted into it normal location on chromosome 5 (MATa can1).

Generating the Double Mutants by Mating

A mating strategy was used to generate the all of the possible double mutants between hnt2 and the collection of 1,600 bar-coded deletion strains. This approach ensures that enough independent isolates can be tested to maintain the complexity of the library of bar-coded deletion mutants. Specifically, we placed 2,000,000 cells of the bar-coded library into separate mating reactions with 5,000,000 hnt2 mutant cells and 5,000,000 HNT2 wild-type cells. The cells were incubated on sterile nitrocellulose filters, colony-side up, on YPD agar plates at 30 degrees for 16 hours. The cells were then plated on SC-URA media with 200 μg/ml G418 to select for diploids. We obtained 2,000,000 independent diploid colonies from the hnt2 cells and 3,000,000 independent diploid colonies from the wild-type control. Diploid cells (2,000,000 from each of the two pools) were incubated for 2 days on pre-sporulation media at 30 degrees and then the entire sporulating patches were transferred to 5 ml of 1% K acetate, 0.005% Zn acetate and allowed to incubate for an additional two days in a roller drum. The extent of sporulation was determined to be 30% and 8,000,000 cells were subjected to random spore analysis with zymolyase and agitation with glass beads. Haploid spores containing the desired double mutations were selected by plating the cells on SC-URA media containing G418 and canavanine. Because canavanine-resistance is recessive, canavanine selects against diploid cells that did not sporulate. The calculated number of URA+, canavanine-resistant, G418-resistant cells is 8,000,000×30% sporulated×4 spores/tetrad×12.5% of the right genotype, or 1,200,000. 420,000 independent colonies resulting from the sporulated double mutant pool and 380,000 independent colonies from the sporulated single mutant pool were obtained. The fact that the yield was ⅓ of the theoretical yield indicates that some cells are killed treatment with zymolyase and glass beads. However, 400,000 diploids covers the 1,600-fold complex library 250 times. 100 out of 100 of the resulting colonies had an identifiable mating type, proving that the protocol rapidly and efficiently generates haploid strains via mating and random spore analysis.

Competitive Outgrowth to Identify Synthetic Lethal Mutations

Each resulting set of 380,000 to 420,000 independent haploid colonies was pooled and grown for 20 population doublings. Following the outgrowth, 100,000,000 cells from the single mutant control and double mutant pools were harvested and stored at −80 degrees.

Tag Detection

The goal was to identify tags present in the control but missing from the hnt2 deletion strain following the 20 population doublings of competitive outgrowth. These tags represent mutations that are synthetically lethal or synthetically less fit in combination with the hnt2 deletion.

Isolate Genomic DNA from the Surviving Double-Mutants

1. Thaw the hnt2 and control pellets at room temperature. The pellets contain 100,000,000 cells that were harvested after the 20 population doublings of competitive outgrowth.

2. Resuspend pellet in 300 μl Lysis buffer from the Epicentre Kit (MasterPure Yeast DNA Purification Kit #mpy80200).

3. Add acid-washed glass beads (˜0.5 mm) to the meniscus, and vortex for 30 seconds, speed 5000 rpm in the mini-bead beater. Cool on ice immediately.

4. Put tube on ice for 5 mins. Add 150 μl of MPC Protein Precipitation Reagent from the Epicentre Kit. Vortex mix for 10 secs. Microfuge at 14 krpm (or top speed) for 10 mins.

5. Carefully decant supernatant into a fresh Eppendorf tube. Add 500 μl of isopropranol and mix well by inversion. Microfuge at 14 krpm (or top speed) for 10 mins. Wash pellet with 0.5 ml of 70% EtOH, vortex, spin, and pour off the ETOH. Spin for an additional 10 seconds, remove remaining EtOH with a P200 and dry the pellet for 3-5 mins at room temp. Resuspend pellet in 100 μl TE.

6. Quantitate the genomic DNA using the Hoefer DyNA Quant 200 fluorometer.

Tag Amplification

Each deletion strain was labeled with two independent tags, one upstream and one downstream of the selectable marker. The two tags are termed “uptags” and “dntags”. The uptags and dntags are amplified separately from the genomic samples. This tag amplification procedure has been previously described (Winzeler E A, et al., 1999, Science 285:901-6, Giaever G, et al., 1999, Nat Genet 21(3):278-83; Shoemaker D D, et al, 1996, Nat Genet 14(4):450-6). All the “uptag” bar-codes can be amplified using a single pair of common 18mer primers called 1up and 3up (see below). The 3 up primer has a Cy3 or Cy5 label on the 5′ end. The “downtags” bar-codes are flanked by different common priming sites. These tags can be amplified with the two 18mer primers called 1DN and 3DN. Again, the 1DN primer has a Cy3 or Cy5 label on the 5′ end. In this example, the tags from the control cells were amplified with the Cy3 labeled primers and the tags from the hnt2 cells with the Cy5 labeled primers.

5 μl Primer mix (0.5 μM final)

1 μl Genomic template (15 ng final)

44 μl PCR Super Mix (PCR Platinum SuperMix, Gibco # 11306-016)

50 μl total

| UPTAG primer mix: | |
|---|---|
| 5 μM 1UP | 5′ gatgtccacgaggtctct 3′ |
| 5 μM 3UP-Cy3/5 | 5′ Cy-gtcgacctgcagcgtacg 3′ |
| 1UP | |
| 5′ gatgtccacgaggtctct3′ 20mer barcode | |
| 5′ gatgtccacgaggtctctTTGGTGCGCCCACAAACAAAcgtacgctgcaggtcgac -kan | |
| 3′ ctacaggtgctccagagaAACCACGCGGGTGTTTGTTTgcatgcgacgtccagct - kan | |
| 3′ gcatgcgacgtccagctg Cy 5′ | |
| 3UP-Cy | |
| DNTAG primer mix: | |
| 5 μM 1DN-Cy3/5 | 5′ Cy-cgagctcgaattcatcg 3′ |
| 5 μM 3DN | 5′ cggtgtcggtctcgtag 3′ |
| 1DN-Cy | |

```
5′ Cy-cgagctcgaattcatcg 3′ 20mer barcode
kan cgagctcgaattcatcgatTTTCTATATTGGGACACGGGctacgagaccgacacg 3′
kan gctcgagcttaagtagctaAAAGATATAACCCTGTGCCCgatgctctggctgtggc 5′
                                                                                    3′ gatgctctggctgtggc 5′
                                                                                                          3DN
```

PCR reaction conditions:

1. 94C 5 min

2. 94C 30 sec |

3. 50C 30 sec | 35 cycles of conditions 2, 3 and 4.

4. 72C 30 sec |

5. 72C 7min

Hybridizations

The amplified tags are mixed with blocking primers that bind to the common priming sites. This serves of the reduce the background during the hybridization.

1. Transfer 35 μl of the Cy3 and Cy5 uptag amplifications to a 200 μl PCR tube.

2. Add 5 μl of the “upblocking mix” (1UP and 2UP, 100 pmoles each—see below).

3. Transfer 35 μl of the Cy3 and Cy5 downtag amplifications to a separate 200 μl PCR tube.

4. Add 5 μl of the “downblocking mix” (3DN and 4DN, 100 pmoles each—see below)

5. Incubate at 99C for 2 minutes to denature the PCR products.

```
                      1UP 2UP
5′ gatgtccacgaggtctct3′ 20mer barcode 5′cgtacgctgcaggtcgac 3′
3′ ctacaggtgctccagagaAACCACGCGGGTGTTTGTTTgcatgcgacgtccagctg Cy 5′
                                                          20mer barcode
5′ Cy cgagctcgaattcatcgatTTTCTATATTGGGACACGGGctacgagaccgacaccg 3′
        3′gctcgagcttaagtagcta 5′ 3′gatgctctggctgtggc 5′
                          4DN 3DN
```

6. Prepare 3.5 ml of hybridization mix (1M NaCl, 10 mM Tris pH 7.0, 0.5% Triton-X 100).

7. Add the denatured uptags and downtags (75 μl each) to the 3.5 ml hybridization mix.

8. Place a 1×3 glass slide in plastic bag and add the hybridization mix. This 1×3 glass slide contains oligonucleotides that are complementary tag sequences from each of the different deletion strains. In this example, we generated the high-density oligonucleotide array using ink-jet synthesizer developed at Rosetta Inpharmatics (Marton M J, et al., 1988, Nat Med 4(11):1293-301). This array contains the tags for each of the 6,200 deletion stains even though the current pool only contains 1,600 deletion strains. Similar chips can be obtained from Affymetrix Inc. (Santa Clara, Calif.).

9. Open tubes of hybstrip I and transfer to a 15 ml falcon tube containing hybridization mix.

10. Transfer the entire mix to a plastic bag containing a 1×3 cm slide, seal, and place on the roller at 40C for 3 hours.

Washing

1. Add 300 ml of 6×SSPE+0.05% Triton-X to a 300 ml beaker.

2. Remove the chip from the bag and wash with 20 brisk rotations using blue clamps.

3. Add 300 ml of cold 0.06×SSPE to a different 300 ml beaker.

4. Dip the slide in the low salt buffer of step (3).

Scanning

Insert the 1×3 slide into the GMS 418 Array Scanner. This scanner is commercially available from Genetic Microsystems (34 Commerce Way, Woburn, Mass. 01801; http://www.arrayer.com/products/html/maas.html). The slide was scanned in both the Cy5 and Cy3 channels at the appropriate PMT setting. (Winzeler E A, et al., 1999, Science 285:901-6).

Data Analysis

The scanned image of the oligonucleotide array was then analyzed by a standard software package. This program quantifies the signal intensity for each of the different tags in the both the Cy5 and Cy3 channel. After normalizing the data, the program generates ratios for each of the different tags. The final output is a list of the deletion strains in the pool along with the corresponding uptag and downtag ratios. Sorting the list by the ratios in ascending order identifies deletion strains that are synthetically lethal with HNT2. In this example, 1% of the 1,600 tagged deletion strains displayed significant growth differences in the hnt2 genetic background relative to the wild-type control.

Various references, including patent applications, patents, and literature publications are cited herein, the disclosures of which are incorporated by reference in their entireties.

The invention being thus described, it will be obvious that the same may be varied in many ways. Such variation are not to be regarded as a departure from the spirit and scope of the invention, and all such modifications, as would be obvious to a person skilled in the art, are intended to be included in the scope of the following claims.

What is claimed is:

1. A method of screening for cells having a secondary mutation in a second gene that causes a decreased rate of growth in a cell also having a primary mutation in a first gene, said first and second genes being different, comprising the steps of:
(a) introducing a primary mutation into a first gene in one or more cells present in a library of cells having a secondary mutation in a second gene, said library comprising a population of cells wherein each cell in said population has a different secondary mutation in a different gene;
(b) incubating the cells of step (a) under conditions which would, in the absence of step (a), allow the cells to grow; and
(c) comparing the growth of each cell of step (b) that has the primary mutation with the growth of a control cell without the primary mutation but containing said secondary mutation,
wherein any cell in step (b) that exhibits a decreased rate of growth as compared to the rate of growth of a control cell without the primary mutation but containing said secondary mutation is identified as containing a secondary mutation that causes a decreased rate of growth when combined with the primary mutation in a cell.

2. The method of claim 1, wherein the library of mutated cells is a yeast cell library.

3. The method of claim 2, wherein the library is a barcoded deletion mutation library.

4. The method of claim 1, wherein the library of mutated cells is a mammalian cell library.

5. The method of claim 4, wherein the library is a barcoded deletion mutation library.

6. The method of claim 3 or claim 5, wherein the library consists of cells bearing between 100 and 10,000 different mutations, each cell bearing only one mutation.

7. The method of claim 3, wherein the primary mutation is introduced by mating cells bearing the primary mutation with the mutated cells in the library.

8. The method of claim 3, wherein the primary mutation is introduced by direct transformation of the mutated cells in the library.

9. The method of claim 3, wherein the growth of the cells is determined by quantitatively detecting the presence of the barcodes.

10. The method of claim 9, wherein the method of detecting the barcodes comprises amplifying said barcodes by the polymerase chain reaction and hybridizing the products of said reaction to a DNA microarray comprising DNA molecules complementary to one or more of the barcodes.

11. The method of claim 10, wherein the products of the polymerase chain reaction are fluorescently labeled.

12. The method of claim 11, wherein the products of the polymerase chain reaction generated from the control cells is labeled with a different fluorophore as compared to the products of the polymerase chain reaction generated from the cells of step (a), and wherein the comparison of step (c) is performed by comparing the relative amounts of each fluorophore detected at each address on the DNA microarray.

13. A method of screening for and identifying mutated genes that, when combined with a primary mutation in a first gene in a cell, cause a decreased rate of growth of the cell, comprising the steps of:
(a) introducing a primary mutation into a first gene in one or more cells present in a library of cells having a secondary mutation in a second gene, said library comprising a population of cells wherein each cell in said population has a different secondary mutation in a different gene;
(b) incubating the cells of step (a) under conditions which would, in the absence of step (a), allow the cells to grow; and
(c) comparing the growth of each cell of step (b) that has the primary mutation with the growth of a control cell without the primary mutation but containing said secondary mutation,
(d) identifying any cell in step (b) that exhibits a decreased rate of growth as compared to the rate of growth of a control cell without the primary mutation but containing said secondary mutation as containing a secondary mutation that causes a decreased rate of growth when combined with the primary mutation in a cell; and
(e) determining in which gene the secondary mutation that causes a decreased rate of growth when combined with the primary mutation identified in step (d) resides.

14. The method of claim 13, wherein the library of mutated cells is a yeast cell library.

15. The method of claim 14, wherein the library is a barcoded deletion mutation library.

16. The method of claim 13, wherein the library of mutated cells is a mammalian cell library.

17. The method of claim 16, wherein the library is a barcoded deletion mutation library.

18. The method of claim 15 or claim 17, wherein the library consists between 100 and 10,000 different mutant strains.

19. The method of claim 15, wherein the primary mutation is introduced by mating cells bearing the primary mutation with the mutated cells in the library.

20. The method of claim 15, wherein the primary mutation is introduced by direct transformation of the mutated cells in the library.

21. The method of claim 15, wherein the growth of the cells is determined by quantitatively detecting the presence of the barcodes.

22. The method of claim 21, wherein the method of detecting the barcodes comprises amplifying said barcodes by the polymerase chain reaction and hybridizing the products of said reaction to a DNA microarray comprising DNA molecules complementary to one or more of the barcodes.

23. The method of claim 22, wherein the products of the polymerase chain reaction are fluorescently labeled.

24. The method of claim 23, wherein the products of the polymerase chain reaction generated from the control cells is labeled with a different fluorophore as compared to the products of the polymerase chain reaction generated from the cells of step (a), and wherein the comparison of step (c) is performed by comparing the relative amounts of each fluorophore detected at each address on the DNA microarray.

25. The method of claim 13, which further comprises isolating the gene in which the secondary mutation that causes a decreased rate of growth when combined with the primary mutation identified in step (d) resides.

26. The method of claim 15, which further comprises the step of:
(f) isolating the gene in which the secondary mutation that causes a decreased rate of growth when combined with the primary mutation identified in step (d) resides.

27. The method of claim 26 which further comprises the step of:
(g) isolating the human homolog of the gene isolated in step (f).

28. A barcoded deletion mutant library comprising a population of different mutant cells, each mutant cell in said population having a different deletion mutation in a different gene and a different barcode associated therewith, wherein between 25% and 100% of the cells comprising the library also have a primary mutation, wherein said primary mutation is a mutation of the same gene in each of the different mutant cells containing said primary mutation.

29. The barcoded deletion mutant library of claim 28, wherein between 50% and 95% of the cells comprising the library also have a primary mutation, wherein said primary mutation is a mutation of the same gene in each of the different mutant cells containing said primary mutation.

30. The barcoded deletion mutant library of claim 28, wherein between 60% and 90% of the cells comprising the library also have a primary mutation, wherein said primary mutation is a mutation of the same gene in each of the different mutant cells containing said primary mutation.


SEQUENCE LISTING (10 sequences)

SEQ ID NO: 1 - DNA, 18 bp, Artificial Sequence
  primer
gatgtccacgaggtctct

SEQ ID NO: 2 - DNA, 18 bp, Artificial Sequence
  primer
gtcgacctgcagcgtacg

SEQ ID NO: 3 - DNA, 56 bp, Artificial Sequence
  primer
gatgtccacgaggtctctttggtgcgcccacaaacaaacgtacgctgcaggtcgac

SEQ ID NO: 4 - DNA, 56 bp, Artificial Sequence
  primer
gtcgacctgcagcgtacgtttgtttgtgggcgcaccaaagagacctcgtggacatc

SEQ ID NO: 5 - DNA, 17 bp, Artificial Sequence
  primer
cgagctcgaattcatcg

SEQ ID NO: 6 - DNA, 17 bp, Artificial Sequence
  primer
cggtgtcggtctcgtag

SEQ ID NO: 7 - DNA, 56 bp, Artificial Sequence
  primer
cgagctcgaattcatcgattttctatattgggacacgggctacgagaccgacaccg

SEQ ID NO: 8 - DNA, 56 bp, Artificial Sequence
  primer
cggtgtcggtctcgtagcccgtgtcccaatatagaaaatcgatgaattcgagctcg

SEQ ID NO: 9 - DNA, 18 bp, Artificial Sequence
  primer
cgtacgctgcaggtcgac

SEQ ID NO: 10 - DNA, 19 bp, Artificial Sequence
  primer
atcgatgaattcgagctcg